SEQUENCE LISTING (12 sequences)

SEQ ID NO: 1 - protein, 458 aa, Stevia rebaudiana
MENKTETTVHRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFTQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFPWLDQQPSRSVLYVSFGSTSEVDEEDFLEIARGLVDSKQSFLWVVRPGFVKGSTWVDPLPDGFLGERERMMKWVRQQNVLAHGTIGAFWHHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 2 - DNA, 1374 bp, Stevia rebaudiana
  source 1..1374: mol_type = genomic DNA
atggaaaataaaacggagaccaccgttcaccggcgccggagaataatattattcccggtaccatttcaaggccacattaacccaattcttcagctagccaatgtgttgtactccaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcaccgatgctctttggtacttcacgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttccatggttagaccaacaaccgtcacgttcggtactgtatgttagttttggtagtactagtgaagtggatgaggaagatttcttggaaatagctcgtgggttggttgatagcaagcagtcgtttttatgggtggttcgacctgggtttgtcaagggttcgacgtgggtcgacccgttgccagatgggttcttgggtgaaagagaacgtatgatgaaatgggttcggcagcagaatgtgctagctcatggaacaataggcgcattctggcatcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttg

SEQ ID NO: 3 - DNA, 39 bp, synthetic construct
  source 1..39: mol_type = other DNA
acctgccgacgcatggttggttagcgggtttgcgtattc

SEQ ID NO: 4 - DNA, 39 bp, synthetic construct
  source 1..39: mol_type = other DNA
gaatacgcaaacccgctaaccaaccatgcgtcggcaggt

SEQ ID NO: 5 - DNA, 40 bp, synthetic construct
  source 1..40: mol_type = other DNA
catggtccgttagcgggtgtgcgtattccgatcattaacg

SEQ ID NO: 6 - DNA, 40 bp, synthetic construct
  source 1..40: mol_type = other DNA
cgttaatgatcggaatacgcacacccgctaacggaccatg

SEQ ID NO: 7 - DNA, 42 bp, synthetic construct
  source 1..42: mol_type = other DNA
gcctgatcacggatgcgttctggtattttgcacagagcgttg

SEQ ID NO: 8 - DNA, 42 bp, synthetic construct
  source 1..42: mol_type = other DNA
caacgctctgtgcaaaataccagaacgcatccgtgatcaggc

SEQ ID NO: 9 - DNA, 42 bp, synthetic construct
  source 1..42: mol_type = other DNA
cattaaaagcgcatatagccactggcagattgccaaagagat

SEQ ID NO: 10 - DNA, 42 bp, synthetic construct
  source 1..42: mol_type = other DNA
atctctttggcaatctgccagtggctatatgcgcttttaatg

SEQ ID NO: 11 - DNA, 42 bp, synthetic construct
  source 1..42: mol_type = other DNA
gatctttagcgacttcggcatcgaccagccgttgaatgcgcg

SEQ ID NO: 12 - DNA, 42 bp, synthetic construct
  source 1..42: mol_type = other DNA
cgcgcattcaacggctggtcgatgccgaagtcgctaaagatc

(19) United States
(12) Patent Application Publication
(10) Pub. No.: US 2026/0092262 A1
(43) Pub. Date: Apr. 2, 2026

(54) GLUCOSYLTRANSFERASE MUTANT CATALYZING REBAUDIOSIDE D TO PRODUCE REBAUDIOSIDE M AND USE THEREOF

(71) Applicant: Shandong Benyue Biological Technology Co., Ltd, Dongying (CN)

(72) Inventors: Shunbin ZHANG, Dongying (CN); Liang CHEN, Dongying (CN); Zhen ZHANG, Dongying (CN); Boqing DING, Dongying (CN); Jinpeng JIAO, Dongying (CN)

(73) Assignee: Shandong Benyue Biological Technology Co., Ltd, Dongying (CN)

(21) Appl. No.: 19/340,916

(22) Filed: Sep. 26, 2025

(30) Foreign Application Priority Data
Sep. 29, 2024 (CN) 202411366033.8

(51) Int. Cl. C12N 9/10; C12N 15/70; C12P 19/18; C12P 19/44

(52) U.S. Cl. CPC C12N 9/1051; C12N 15/70; C12P 19/18; C12P 19/44; C12N 2800/101

(57) ABSTRACT

A glucosyltransferase mutant catalyzing RD to produce RM and a use thereof are provided. The glucosyltransferase mutant is obtained by subjecting the amino acid sequence shown in SEQ ID NO: 1 to at least one of the following mutations: mutating proline at position 84 to tryptophan; and/or mutating methionine at position 88 to valine; and/or mutating leucine at position 126 to phenylalanine; and/or mutating asparagine at position 196 to histidine; and/or mutating leucine at position 379 to isoleucine. The use of the mutant in preparation of the RM is also provided. The enzyme activity of the mutant is significantly higher than that of the wild type, and the conversion rate for the preparation of the RM is high.

### CROSS REFERENCE TO THE RELATED APPLICATIONS

This application is based upon and claims priority to Chinese Patent Application No. 202411366033.8, filed on Sep. 29, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBFSTZB032-PKG_SequenceListing.xml, created on Sep. 10, 2025, and is 13,529 bytes in size.

### TECHNICAL FIELD

The present invention belongs to the technical field of enzyme engineering and specifically relates to a glucosyltransferase mutant catalyzing rebaudioside D (RD) to produce rebaudioside M (RM) and use thereof.

### BACKGROUND

Steviol glycosides (SGs) include stevioside, rebaudioside A (RA), rebaudioside B (RB), rebaudioside C (RC), RD, rebaudioside E (RE), rebaudioside F (RF), dulcoside A, steviolbioside, RM, and rebaudioside I (RI). RM accounts for 0.3%-1.0% of the total content of the SGs, and possesses the advantages of the SGs, such as low calories, high sweetness, and a pure taste. It is the component in the SGs that has a taste closest to sucrose, with a relative sweetness 200-350 times that of the sucrose. RM is sweeter than the mainstream RA and less bitter than RA. Therefore, the RM component of the SGs in Stevia rebaudiana has a wider range of applications. It is suitable for use as a tabletop sweetener and in most foods, beverages, and other products. Additionally, it is calorie-free and contains no harmful chemicals to the human body and the environment, making it an excellent choice for patients with diabetes or other health issues. As a sucrose substitute, RM has significant application value in the food industry, reducing demand for traditional sweeteners and helping to alleviate the environmental pressures associated with their production, including impacts on land, water resources, and greenhouse gas emissions. It also contributes to maintaining the health of ecosystems, including the oceans.

Traditional methods for extracting the SGs mostly include plant extraction methods, such as hot water extraction, solvent extraction, immersion, macroporous resin adsorption, and microwave-assisted extraction. These methods not only have disadvantages such as low efficiency and waste of water, but also involve high cost and the yield being unable to meet market demand. The biotransformation of the SGs is currently the most economical and effective way to achieve industrial production of the SGs. The biotransformation of the SGs mainly includes: (1) constructing a SG synthesis pathway by expressing glycosyltransferase genes in microorganisms to heterologously synthesize the SGs using glucose as a carbon source; (2) synthesizing the SGs using biocatalysis. Biocatalytic synthesis of the SGs refers to the process of synthesizing the SGs using enzymes or enzyme-producing microorganisms as catalysts.

Currently, the biosynthesis of RM mainly focuses on the modification of UGT76G1 derived from Stevia rebaudiana. Chinese invention patents CN109750071A, CN114214378A, and CN114574460A all have conducted research on this gene, but the currently available target proteins are still limited. The present invention is based on the protein structure and experimental data to perform rational modifications of the protein from the perspective of molecular interactions, thereby obtaining a mutant with enhanced enzyme activity, which can be effectively applied in catalytic conversion. A conversion rate of 95% can be achieved, which is of great significance for the industrial production of RM of the SGs.

### SUMMARY

The technical problem to be solved by the present invention is to provide a glucosyltransferase mutant catalyzing RD to produce RM, and a use thereof in preparation of the RM. An enzyme activity of the mutant is significantly higher than that of the wild type, and a conversion rate for the preparation of RM is high.

The technical solution of the present invention is as follows:

The glucosyltransferase mutant catalyzing the RD to produce the RM, wherein the amino acid sequence of a glucosyltransferase is as shown in SEQ ID NO: 1, and the glucosyltransferase mutant is obtained by subjecting the amino acid sequence shown in SEQ ID NO: 1 to at least one of the following mutations:
mutating proline at position 84 to tryptophan, designated as P84W;
and/or mutating methionine at position 88 to valine, designated as M88V;
and/or mutating leucine at position 126 to phenylalanine, designated as L126F;
and/or mutating asparagine at position 196 to histidine, designated as N196H;
and/or mutating leucine at position 379 to isoleucine, designated as L379I.

The glucosyltransferase is derived from Stevia rebaudiana, and the nucleotide sequence of the glucosyltransferase is as shown in SEQ ID NO: 2.

The amino acid sequence of the glucosyltransferase is as shown in SEQ ID NO: 1:

MENKTETTVHRRRRIILFPVPFQGHINPILQLANVLYSKGFSITIFHTNF
NKPKTSNYPHFTFRFILDNDPQDERISNLPTHGPLAGMRIPIINEHGADE
LRRELELLMLASEEDEEVSCLITDALWYFTQSVADSLNLRRLVLMTSSLF
NFHAHVSLPQFDELGYLDPDDKTRLEEQASGFPMLKVKDIKSAYSNWQIL
KEILGKMIKQTKASSGVIWNSFKELEESELETVIREIPAPSFLIPLPKHL
TASSSSLLDHDRTVFPWLDQQPSRSVLYVSFGSTSEVDEEDFLEIARGLV
DSKQSFLWVVRPGFVKGSTWVDPLPDGFLGERERMMKWVRQQNVLAHGTI
GAFWHHSGWNSTLESVCEGVPMIFSDFGLDQPLNARYMSDVLKVGVYLEN
GWERGEIANAIRRVMVDEEGEYIRQNARVLKQKADVSLMKGGSSYESLES
LVSYISSL.

The nucleotide sequence of the glucosyltransferase is as shown in SEQ ID NO: 2:

atggaaaata aaacggagac caccgttcac cggcgccgga
gaataatatt attcccggta ccatttcaag gccacattaa
cccaattctt cagctagcca atgtgttgta ctccaaagga
ttcagtatca ccatctttca caccaacttc aacaaaccca
aaacatctaa ttaccctcac ttcactttca gattcatcct
cgacaacgac ccacaagacg aacgcatttc caatctaccg
actcatggtc cgctcgctgg tatgcggatt ccgattatca
acgaacacgg agctgacgaa ttacgacgcg aactggaact
gttgatgtta gcttctgaag aagatgaaga ggtatcgtgt
ttaatcaccg atgctctttg gtacttcacg caatctgttg
ctgacagtct taacctccga cggcttgttt tgatgacaag
cagcttgttt aattttcatg cacatgtttc acttcctcag
tttgatgagc ttggttacct cgatcctgat gacaaaaccc
gtttggaaga acaagcgagt gggtttccta tgctaaaagt
gaaagacatc aagtctgcgt attcgaactg gcaaatactc
aaagagatat tagggaagat gataaaacaa acaaaagcat
cttcaggagt catctggaac tcatttaagg aactcgaaga
gtctgagctc gaaactgtta tccgtgagat cccggctcca
agtttcttga taccactccc caagcatttg acagcctctt
ccagcagctt actagaccac gatcgaaccg tttttccatg
gttagaccaa caaccgtcac gttcggtact gtatgttagt
tttggtagta ctagtgaagt ggatgaggaa gatttcttgg
aaatagctcg tgggttggtt gatagcaagc agtcgttttt
atgggtggtt cgacctgggt ttgtcaaggg ttcgacgtgg
gtcgacccgt tgccagatgg gttcttgggt gaaagagaac
gtatgatgaa atgggttcgg cagcagaatg tgctagctca
tggaacaata ggcgcattct ggcatcatag cggatggaac
tctacgttgg aaagcgtttg tgaaggtgtt cctatgattt
tctcggattt tgggctcgat caaccgttga atgctagata
catgagtgat gttttgaagg taggggtgta tttggaaaat
gggtgggaaa gaggagagat agcaaatgca ataagaagag
ttatggtgga tgaagaagga gaatacatta gacagaatgc
aagagttttg aaacaaaagg cagatgtttc tttgatgaag
ggtggttcgt cttacgaatc attagagtct ctagtttctt
acatttcatc gttg.

The present invention provides a use of the glucosyltransferase mutant in preparation of the RM.

A method for preparing the RM includes the following steps:
(1) preparing an enzyme solution of the glucosyltransferase mutant; and
(2) using the RD as a substrate, adding sucrose and sucrose synthase, then adding the enzyme solution prepared in the step (1), and performing a catalytic reaction to obtain the RM. Catalytic reaction conditions are as follows: a pH value is 6-7, and a temperature is 37-38° C. Addition amounts of RD, the sucrose, the sucrose synthase, and the enzyme solution can be adjusted according to actual needs.

In the step (1), a method for preparing the enzyme solution of the glucosyltransferase mutant includes the following steps:
a, designing and synthesizing site-directed mutagenesis primers based on a nucleotide sequence of the glucosyltransferase, performing site-directed mutagenesis on a glucosyltransferase gene, and constructing a plasmid vector carrying the mutant gene;
b, transforming the plasmid vector into a host cell; and
c, selecting positive clones for culture to obtain the enzyme solution of the glucosyltransferase mutant.

Preferably, the plasmid vector is a pET vector.

Preferably, the host cell is a bacterial cell or a fungal cell, more preferably Bacillus subtilis or Escherichia coli.

Compared with the prior art, the present invention possesses the following advantages:

The glucosyltransferase mutants P84W, M88V, L126F, N196H, and L379I provided in the present invention exhibit shake flask fermentation enzyme activities that are 1.12 times, 1.69 times, 1.62 times, 1.63 times, and 1.29 times higher than that of the wild type, respectively. The present invention also provides the use of glucosyltransferase mutants in catalyzing the synthesis of RM from RD, achieving a conversion rate of 95%. The glucosyltransferase mutants provided by the present invention enhance the application potential of glucosyltransferases in the food, pharmaceutical, and chemical industries, and are of great significance for the industrial production of glucosyltransferases.

### BRIEF DESCRIPTION OF THE DRAWINGS

FIG. 1 shows the high-performance liquid chromatography (HPLC) chromatogram of the enzyme activity of the glucosyltransferase mutant M88V.

FIG. 2 shows the HPLC chromatogram of the catalytic product of the enzyme solution of the glucosyltransferase mutant M88V.

### DETAILED DESCRIPTION OF THE EMBODIMENTS

The examples of the present invention are intended only to further illustrate the content of the present invention and are not intended to limit its content or scope. For molecular biology experimental methods not specifically described in the present examples, please refer to Molecular Cloning: A Laboratory Manual.

The culture media used in the examples are as follows:
Luria-Bertani (LB) medium: 10 g/L tryptone, 5 g/L yeast extract, 10 g/L sodium chloride;
Terrific Broth (TB) medium: 12 g/L tryptone, 24 g/L yeast extract, 5 g/L glycerol, 2.31 g/L KH₂PO₄, 16.43 g/L K₂HPO₄·3H₂O.

### Example 1: Construction of Wild-Type Glucosyltransferase Strain

The UGT76G1 sequence derived from Stevia rebaudiana was submitted to gene company for synthesis to construct the wild-type strain PET28a-GT. The 1% inoculum of the PET28a-GT bacterial solution was inoculated into LB liquid medium (containing 50 mg/L kanamycin sulfate) to prepare the seed culture. This seed culture was then inoculated into TB liquid medium at the 1% inoculum. Fermentation was induced on the shaking incubator at 20° C. for 48 h. The fermentation broth was then centrifuged at 4° C. and 12,000 rpm for 15 min. The precipitate was collected and crushed, and the supernatant, which was the wild-type Glucosyltransferase enzyme solution, was obtained.

### Example 2: Preparation and Expression of Glucosyltransferase Mutants

Based on the nucleotide sequence of the glucosyltransferase, site-directed mutagenesis primers for introducing single mutations were designed and synthesized. Site-directed mutagenesis was performed on the glucosyltransferase gene. The plasmid vector carrying the mutant gene was constructed using seamless cloning technology, where the plasmid vector used was pET28a(+). The vector was introduced into Escherichia coli BL21-DE3 for induced expression, resulting in the glucosyltransferase mutant strains. Site-directed mutagenesis: The target gene was subjected to site-directed mutagenesis using gene overlap-extension polymerase chain reaction (PCR). The linearized fragment was recovered and purified for recombination.

The nucleotide sequences of the site-directed mutagenesis primers are as follows:

The site-directed mutagenesis primers for introducing the P84W mutation are:
Forward primer: 5′-ACCTGCCGACGCATGGTTGGTTAGCGGGTTTGCGTATTC-3′, as shown in SEQ ID NO: 3;
Reverse primer: 5′-GAATACGCAAACCCGCTAACCAACCATGCGTCGGCAGGT-3′, as shown in SEQ ID NO: 4.

The site-directed mutagenesis primers for introducing the M88V mutation are:
Forward primer: 5′-CATGGTCCGTTAGCGGGTGTGCGTATTCCGATCATTAACG-3′, as shown in SEQ ID NO: 5;
Reverse primer: 5′-CGTTAATGATCGGAATACGCACACCCGCTAACGGACCATG-3′, as shown in SEQ ID NO: 6.

The site-directed mutagenesis primers for introducing the L126F mutation are:
Forward primer: 5′-GCCTGATCACGGATGCGTTCTGGTATTTTGCACAGAGCGTTG-3′, as shown in SEQ ID NO: 7;
Reverse primer: 5′-CAACGCTCTGTGCAAAATACCAGAACGCATCCGTGATCAGGC-3′, as shown in SEQ ID NO: 8.

The site-directed mutagenesis primers for introducing the N196H mutation are:
Forward primer: 5′-CATTAAAAGCGCATATAGCCACTGGCAGATTGCCAAAGAGAT-3′, as shown in SEQ ID NO: 9;
Reverse primer: 5′-ATCTCTTTGGCAATCTGCCAGTGGCTATATGCGCTTTTAATG-3′, as shown in SEQ ID NO: 10.

The site-directed mutagenesis primers for introducing the L379I mutation are:
Forward primer: 5′-GATCTTTAGCGACTTCGGCATCGACCAGCCGTTGAATGCGCG-3′, as shown in SEQ ID NO: 11;
Reverse primer: 5′-CGCGCATTCAACGGCTGGTCGATGCCGAAGTCGCTAAAGATC-3′, as shown in SEQ ID NO: 12.

PCR reaction system: 1 μL each of 10 μM forward and reverse primers, 25 μL PrimeStar polymerase, 1 μL template, and adding ddH₂O to bring the final volume to 50 μL.

PCR reaction procedure: 94° C. pre-denaturation for 4 min; followed by 25 cycles (94° C. for 10 s, 55° C. for 5 s, 72° C. for 7 min 50 s); extension at 72° C. for 10 min; and hold at 4° C. at the end.

PCR products were verified by 1% agarose gel electrophoresis.

The verified PCR product was seamlessly cloned and transformed into E. coli TOP10 competent cells. The transformation product was plated on LB plates containing 50 mg/L kanamycin sulfate and cultured overnight at 37° C. Five single colonies were picked from the plates and inoculated into LB liquid medium. The culture was verified by PCR and sequenced, and the results were correct.

The plasmid verified as correct by sequencing was transformed into E. coli BL21 (DE3) to generate recombinant E. coli expressing the mutant.

### Mutant Expression:

The prepared recombinant E. coli was inoculated into LB liquid medium and cultured for 16 h to obtain the seed culture. The seed culture was then inoculated into TB liquid medium at the 1% inoculum. The E. coli was cultured and fermented in the shaking incubator at 20° C. for 48 h. The fermentation broth was centrifuged at 4° C. and 12,000 rpm for 15 min and crushed, and the supernatant was obtained as the glucosyltransferase mutant enzyme solution.

### Example 3: Enzyme Activity Assay of Glucosyltransferase Mutants

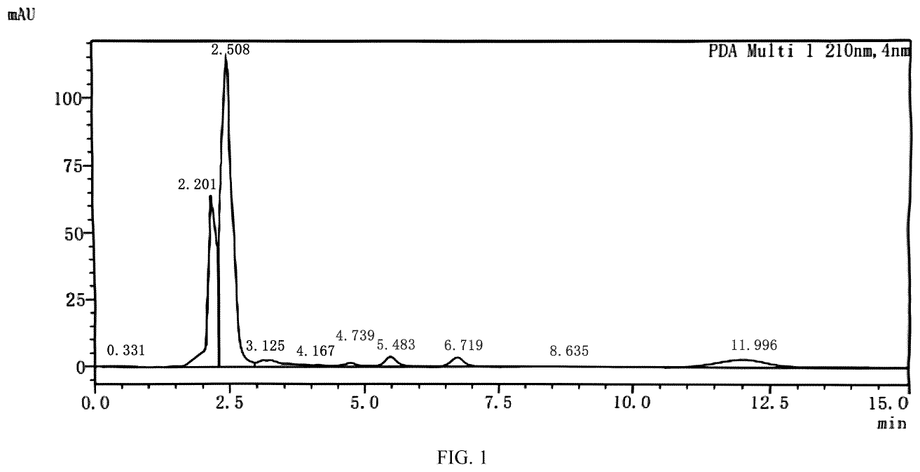

The enzyme solution obtained in Example 2 was assayed for activity. The enzyme activity assay method was as follows: 1 mL of 1.2 mM RD, 1 mL of uridine diphosphoglucose (UDPG), and 1 mL of wild-type or mutant glucosyltransferase enzyme solution were reacted at 37° C. and 200 rpm for 1 h. The changes in the concentrations of substrate RD and product RM were then determined by HPLC assay. The enzyme activity results for the wild-type and mutant glucosyltransferases are shown in Table 1 (wild-type enzyme activity is considered 100%). The HPLC chromatogram of the enzyme activity of mutant M88V is shown in FIG. 1.

The specific HPLC assay method parameters are as follows: adding 200 μL of chromatography-grade acetonitrile, shaking to mix well, letting stand for 10 min, centrifuging at 12,000 rpm for 10 min at room temperature, and passing the supernatant through the 0.22 μm organic membrane for analysis. HPLC was performed using the C18 reverse-phase bonded silica gel separation column (4.6 mm×250 mm, 5 μm) with 25% acetonitrile as the mobile phase at the flow rate of 1 mL/min and the column temperature of 40° C. The UV-detector photodiode array (PDA) was used at the PDA detector wavelength of 210 nm, and the injection volume was 10 μL.

TABLE 1
Enzyme activity assay results for wild-
type and mutant glucosyltransferases

| Sample | Retention Time | Peak | Enzyme Activity |
|---|---|---|---|
| Name | (min) | Area | (%) |
| M6 (wild type) | 6.719 | 65142 | 100 |
| P84W | 6.723 | 72897 | 111.9047619 |
| M88V | 6.713 | 109989 | 168.8449848 |
| L126F | 6.717 | 105641 | 162.1703356 |
| N196H | 6.7 | 105857 | 162.5019189 |
| L379I | 6.704 | 84043 | 129.0150748 |

As shown in Table 1, the enzyme activities of the glucosyltransferase mutants provided in the present invention are all higher than that of the wild type. The shake flask fermentation enzyme activities of mutants P84W, M88V, L126F, N196H, and L379I were 1.12 times, 1.69 times, 1.62 times, 1.63 times, and 1.29 times higher than that of the wild type, respectively.

### Example 4: Conversion of Glucosyltransferase

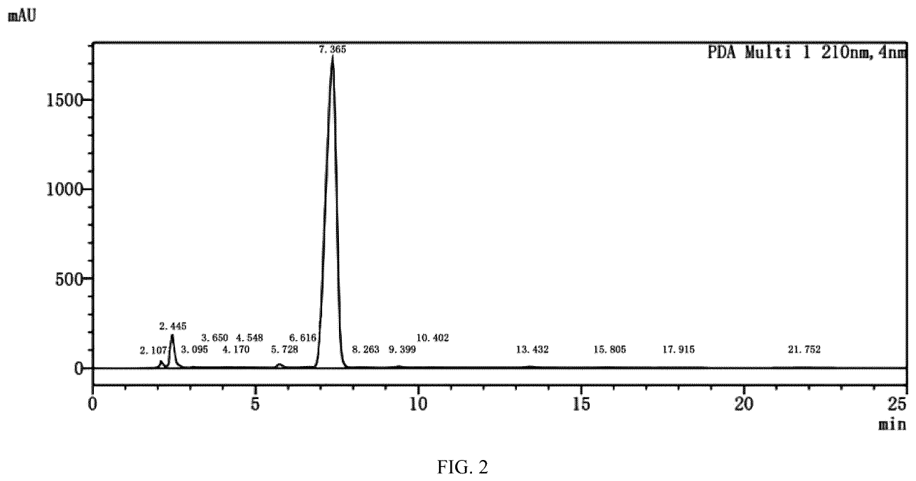

The enzyme solution of mutant M88V obtained in Example 2 was used in the catalytic reaction: water was used as the solvent, 30 g/L of RD, 30 g/L of sucrose, and 5 g/L of sucrose synthase were added, followed by 8 g/L of mutant M88V enzyme solution. The catalytic reaction was carried out at 37° C. and pH 7 for 24 h. After completion of the reaction, HPLC assay was performed. The results are shown in FIG. 2, indicating the conversion rate of 95% after 24 h.

1. A glucosyltransferase mutant catalyzing rebaudioside D (RD) to produce rebaudioside M (RM), wherein the amino acid sequence of a glucosyltransferase is as shown in SEQ ID NO: 1, and the glucosyltransferase mutant is obtained by subjecting the amino acid sequence shown in SEQ ID NO: 1 to the following mutation:
mutating asparagine at position 196 to histidine.

2. The glucosyltransferase mutant catalyzing the RD to produce the RM according to claim 1, wherein the glucosyltransferase is derived from Stevia rebaudiana, and the nucleotide sequence of the glucosyltransferase is as shown in SEQ ID NO: 2.

3. A use of the glucosyltransferase mutant catalyzing the RD to produce the RM according to claim 1 in preparation of the RM.

4. The use according to claim 3, wherein a method for preparing the RM comprises the following steps:
(1) preparing an enzyme solution of the glucosyltransferase mutant; and
(2) using the RD as a substrate, adding sucrose and sucrose synthase, then adding the enzyme solution prepared in the step (1), and performing a catalytic reaction to obtain the RM.

5. The use according to claim 4, wherein a method for preparing the enzyme solution of the glucosyltransferase mutant comprises the following steps:
a, designing and synthesizing site-directed mutagenesis primers based on a nucleotide sequence of the glucosyltransferase, performing site-directed mutagenesis on a glucosyltransferase gene to obtain a mutant gene, and constructing a plasmid vector carrying the mutant gene;
b, transforming the plasmid vector into a host cell; and
c, selecting positive clones for culture to obtain the enzyme solution of the glucosyltransferase mutant.

6. The use according to claim 5, wherein the plasmid vector is a pET vector.

7. The use according to claim 5, wherein the host cell is a bacterial cell or a fungal cell.

8. The use according to claim 7, wherein the host cell is Bacillus subtilis or Escherichia coli.

9. The use according to claim 4, wherein catalytic reaction conditions in the step (2) are as follows: a pH value is 6-7, and a temperature is 37-38° C.
